(19) United States
(12) Patent Application Publication
(10) Pub. No.: US 2024/0358605 A1
(43) Pub. Date: Oct. 31, 2024

(54) PERSONAL CARE COMPOSITIONS

(71) Applicant: Reckitt Benckiser Health Limited, Slough (GB)

(72) Inventors: Tamika Carty, Montvale, NJ (US); Ashwini Dalvi, Montvale, NJ (US); Sarah Frances De Szalay, West Milford, NJ (US); Jouyeop Lee, Montvale, NJ (US); Han Choi Yew, Montvale, NJ (US)

(21) Appl. No.: 18/762,075

(22) Filed: Jul. 2, 2024

Related U.S. Application Data
(62) Division of application No. 18/254,976, filed on May 30, 2023.
(60) Provisional application No. 63/119,096, filed on Nov. 30, 2020.

(30) Foreign Application Priority Data
Mar. 22, 2021 (GB) 2103952.4

(51) Int. Cl. A61K 8/02; A61K 8/362; A61K 8/365; A61K 8/44; A61K 8/46; A61Q 19/10

(52) U.S. Cl. CPC A61K 8/0208; A61K 8/362; A61K 8/365; A61K 8/442; A61K 8/463; A61Q 19/10; A61K 2800/30; A61K 2800/5424; A61K 2800/5428

(57) ABSTRACT

Personal care compositions comprising non-sulfonate anionic surfactant, amphoteric surfactant, and at least two carboxylic acids or salts thereof, and wherein the compositions contain no more than 30% of the total wt % of a) and b) of sulfonated and do not contain para-chloro-meta-xylenol in an amount above 0.15% wt or salicylic acid in an amount above 0.25% wt. The compositions are intended to provide cleansing and antimicrobial benefits yet be well tolerated by skin and hair. Also provided are a dispenser and disposable wipe containing the composition, the use of the composition to provide an antimicrobial benefit to skin and/or hair, and a method for providing an antimicrobial benefit to skin and/or hair by contact with the composition.

### CROSS-REFERENCE TO RELATED APPLICATIONS

The present application is a divisional of U.S. application Ser. No. 18/254,976, filed May 30, 2023; which is a US national stage entry under 35 USC § 1.371 of International Application No. PCT/BG2021/053055, filed Nov. 24, 2021; which claims priority to U.S. Provisional Application No. 63/119,096, filed Nov. 30, 2020; and GB Application No. 2103952.4, filed Mar. 22, 2021. The entire contents of the above-referenced patent applications are hereby expressly incorporated herein by reference.

### TECHNICAL FIELD

The present disclosure relates to personal care compositions suitable for personal care applications, particularly for application to skin and/or hair to provide a cleansing and/or an antimicrobial effect. More particularly, the compositions according to the present disclosure provide a cleansing and antimicrobial effect on the skin and/or hair. The compositions are preferably well tolerated by the skin yet provide an appreciable antimicrobial benefit.

### BACKGROUND

Compositions for topical application are well-known in the personal care field, including for cosmetic and dermatological use, as well as in the pharmaceutical fields. Topical compositions are intended to provide at least one specific benefit upon use.

For example, liquid personal care compositions which are primarily intended to provide a cleansing or an antimicrobial effect to the area of skin being treated are well known in the field of personal care products.

Consumers are very aware of the health benefits of using antimicrobial products to protect them from undesirable bacteria and viruses and this desire is increasing as the awareness of personal hygiene increases. Typically, such antimicrobial products are used to cleanse and sanitize the user in many different situations. Examples include hand washing, and, washing of other body parts e.g. when bathing and showering. In particular, antimicrobial personal care compositions are especially desirable in situations where hygienic practices may be variable, or, a high standard of hygiene is vital (for example nurseries, educational establishments, hospitals, public toilets and commercial kitchens). The use of personal care compositions which provide an effective antimicrobial and cleansing benefit is therefore important to consumers who seek protection from undesirable bacteria and viruses. This in turn helps to reduce the spread of disease.

### PRIOR ART

Personal care compositions, including those intended for cleansing applications are well known in the literature.

Budhian et al in WO2017/072482 disclose treatment compositions which are to be used to impart an antimicrobial effect to animate and inanimate surfaces being treated. The compositions comprise anionic surfactants and as an antimicrobial constituent at least one of lactic acid, citric acid, tartaric acid, the substituted acids thereof, derivatives thereof or salts thereof.

Bruning et disclose in WO2017/055789 personal wash compositions, particularly female intimate cleansing compositions personal wash compositions. The compositions comprise lactic acid to provide an antimicrobial affect in combination with a surfactant system.

Yuan et al in US 2008/0247960 disclose foaming topical compositions for application to the human body, particularly to the skin, which provide both a cleaning benefit and a durable antimicrobial benefit. The compositions comprise anionic surfactants with salicylic acid and optionally other organic acids including citric acid or lactic acid as an antimicrobial constituent.

Rypkema et al in WO2006/027551 disclose a liquid composition for treatment of the skin and/or hair to provide a cleaning and/or sanitizing effect thereto. The compositions comprise sodium lauryl ether sulphate as surfactant, benzoic acid as a biocidal constituent with sodium lactate and citric acid included as buffers.

WO2015/058942 discloses compositions for liquid cleaning and disinfection which comprise the anionic surfactant SLES and two carboxylic acids, one of which is citric acid. The other is chosen from malonic acid, malic acid or glycolic acid. Comparative examples use citric acid in combination with lactic acid.

Skin or hair care compositions comprising a stabilizing acid, such as lactic acid, are disclosed in EP 1 593 371A1.

WO2020/165566 discloses personal care products, particularly hand sanitizer compositions, which contain anionic sulfate surfactants and citric and/or lactic acid.

De Szalay et al in WO2018/078336 disclose female intimate cleansing compositions comprising lactic acid and an anionic constituent system comprising secondary alkane sulfonate compound(s), an N-acyl sarcosinate compound and an aromatic hydrotrope compound to boost the antimicrobial activity of the lactic acid.

Vermeulen et al in WO2013/101932 disclose antibacterial liquid soap compositions which comprise lactic acid/lactate, surfactants and alcohol.

Liquid cleansing compositions for use on skin and comprising an antibacterial system comprising certain organic acids are disclosed in WO2018/022016.

The use of organic acids for microbiological control is also known in applications outside of personal care applications.

Cornford discloses in WO2013/185074 cleaning and sanitizing compositions which may optionally comprise an anionic surfactant. Other optional ingredients include lactic acid or citric acid. However, no examples with these ingredients are provided.

For example, in WO01/64035, acidic antimicrobial compositions for treating food and food contact surfaces are disclosed. These compositions may comprise anionic surfactant and an organic acid.

WO01/94513 discloses biocidal cleaner compositions which comprise between certain anionic surfactants anionic surfactant and an acid which may be selected from various carboxylic acids.

In WO99/29815 dishwashing compositions comprising a salt of a carboxylic acid for antibacterial activity are disclosed.

Concentrated solutions of acid, which may comprise surfactant, are disclosed for use in the fermentation of hops in WO2015/136366.

### PROBLEMS THE PRESENT DISCLOSURE SEEKS TO ADDRESS

The basic principle of personal washing and of antimicrobial protection (sanitization) is recognized to encompass the need to control, or break, the potential chain of infection from person to person, or, from inanimate object to person. Personal care products for use in personal washing, personal hygiene and personal care applications have been used for many years.

Whilst sanitizing personal care products have been used for many years to sanitize skin there remains a need for such compositions which provide a balance of several desirable characteristics.

As cleansing and sanitizing compositions are often used in situations where frequent sanitization is required, or the user wishes for frequent sanitization, the cumulative effect of this frequent use can be significantly detrimental to the skin. With repeated use of such compositions the treated skin can become irritated and/or dry, sometimes severely. This may lead to the skin become cracked and/or feeling tight or sore. The skin may also become discoloured or appear inflamed. Frequent use of sanitizing personal care compositions on the skin may lead to disruption of the integrity of the skin mantel. This in turn may lead to infection.

Prior art sanitizing compositions, especially those containing ingredients which are not well tolerated by the skin, may irritate and/or dry the skin especially with repeated use. As personal sanitization needs to be repeated frequently in order to maintain sanitization and avoid undesirable levels of microbial growth, it is desirable that personal sanitizing compositions provide for effective sanitization yet are suitable for frequent use as they do not negatively affect the skin and/or hair to which they are applied. Such negative effects may deter use of such sanitizing compositions frequently, or, completely.

Thus, there is a need in the art for cleansing compositions which are well tolerated by the skin (even upon repeated use) and which do not cause unacceptable levels of irritation and/or dryness but which still provide effective levels of cleansing and/or sanitizing, and in certain non-limiting embodiments, effective levels of both cleansing and sanitizing.

There is also a need for sanitizing compositions which users find convenient to use in a variety of situations. Most consumers find sore, irritated and/or dry skin to be inconvenient and uncomfortable. Thus, the provision of a composition which consumers can use to conveniently and effectively cleanse and sanitize their skin and/or hair, especially on a frequent basis, is desirable.

If cleansing and sanitizing compositions are used on hair, the same considerations regarding dryness apply for the hair. Also, the considerations for the skin which is in contact with the hair being treated are as above.

It is also desirable to provide effective levels of germ kill on both gram-positive bacteria (such as S. aureus) and gram-negative bacteria (such as E. coli) in order to effectively sanitize. To achieve effective sanitization, the compositions should ideally provide at least a 3 log10 reduction of various microorganisms/bacteria when tested according to the standard test protocols of at least one of ASTM E2315-03 “Standard Guide for Assessment of Antimicrobial Activity Using a Time-Kill Procedure on gram-positive and/or gram-negative bacteria or EN 1276:2009 method entitled “Chemical Disinfectants and Antiseptics—Quantitative Suspension Test for the Evaluation of bactericidal activity of chemical disinfectants and antiseptics used in food, industrial, domestic and institutional areas—Test method and requirements (Phase 2, step 1)”. Achieving this level of sanitation is typically difficult without including high levels of alcohol and/or strongly biocidal ingredients in a personal care composition.

Furthermore, it is desirable to provide effective antimicrobial action for personal care compositions in contact times which the user will find convenient.

The present disclosure seeks to address one or more of the aforementioned technical problems.

In particular, the present disclosure seeks to provide sanitizing compositions for personal use, which can be effectively used on skin and/or hair to provide cleansing and santization, and which are well tolerated even after repeated use as they do not cause unacceptable levels of detrimental effects to the skin and/or hair such as skin irritation, damage to the skin mantel and/or drying.

It is also an object of the present disclosure to provide sanitizing compositions for personal use which are convenient to use and/or can be used in a variety of situations to encourage frequent use.

It is also an object of the present disclosure to provide sanitizing compositions for personal use, which can especially be used on skin and/or hair and which are effective on both gram-negative bacteria and/or gram-positive bacteria and a particular object is to provide at least a 3 log₁₀ reduction of various microorganisms/bacteria as detailed herein.

It is also an object of the present disclosure to provide sanitizing compositions for personal use, which can especially be used on skin and/or hair and which exhibit good antimicrobial activity within a convenient contact time for the user, e.g. of up to 60 seconds.

Surprisingly, the inventors have found that one or more of the above problems can be ameliorated by compositions according to the present disclosure.

In particular, the present inventors have found that effective cleansing and sanitizing compositions for personal use on skin and/or hair, which can even be used on a frequent basis, are obtained when formulated according to the present disclosure.

The inventors have also found the compositions of the present disclosure are convenient to use. In particular, they do not discourage frequent use as they are well tolerated in personal care applications.

Moreover, the inventors have found that their compositions provide good antimicrobial effects and typically provide at least a 3 log₁₀ reduction of various microorganisms including on both gram-negative and gram-positive bacteria e.g. when tested according to the standard test protocols of at least one of ASTM E2315-03 or EN 1276:2009.

The inventors have also found their compositions to provide effective cleansing and sanitizing effects with treatment times of up to 60 seconds, e.g. up to 30 seconds.

The compositions of the present disclosure are convenient to use.

The present inventors have found that their compositions do not cause unacceptable skin irritation or damage to the skin mantel. It has also been found that the compositions do not unacceptably dry the skin the skin even after repeated use.

### DETAILED DESCRIPTION

Accordingly, in first embodiment of the first aspect of the present disclosure there are provided personal care compositions comprising:
a) 0.1% wt to 20% wt of at least one non-sulfonate anionic surfactant,
b) at least one amphoteric surfactant,
c) at least two carboxylic acids or salts thereof, and
   wherein the at least two carboxylic acids or salts thereof comprises citric acid and the compositions contain no more than 30% of the total wt % of a) and b) of sulfonated compounds, and, further wherein the compositions do not contain para-chloro-meta-xylenol in an amount above 0.15% wt and do not contain salicylic acid in an amount above 0.25% wt.

In a second embodiment of the first aspect of the present disclosure there is provided personal care compositions comprising:
a) at least one non-sulfonate anionic surfactant,
b) at least one amphoteric surfactant,
c) at least two carboxylic acids or salts having different number of carboxylic acid groups, and
   and wherein the compositions comprise a higher % wt of the carboxylic acid having the lowest number of carboxylic acid groups, and no more than 30% of the total wt % of a) and b) of sulfonated compounds, and, further wherein the compositions do not contain para-chloro-meta-xylenol in an amount above 0.15% wt and do not contain salicylic acid in an amount above 0.25% wt.

The following particular (but non-limiting) features apply to both aspects of the present disclosure unless stated otherwise.

In certain particular (but non-limiting) embodiments, the compositions of the present disclosure are liquid compositions.

In certain particular (but non-limiting) embodiments, the compositions of the present disclosure are aqueous compositions.

In certain particular (but non-limiting) embodiments, the compositions of the present disclosure comprise water in an amount of from 65% wt to 99% wt, based on the total weight of the composition.

In certain particular (but non-limiting) embodiments, the non-sulfonate anionic surfactant is selected from the group consisting of alkyl sulfates or salts thereof and alkyl ether sulfates or salts thereof.

In certain particular (but non-limiting) embodiments, the alkyl sulfates or salts thereof are selected from the group consisting of C₈-C₁₈ alkyl sulfates and their salts and C₈-C₁₈ alkyl ether sulfates and their salts.

In certain particular (but non-limiting) embodiments, the alkyl sulfate comprises lauryl sulfate and the alkyl ether sulfate comprises lauryl ether sulfate.

In the second embodiment of the present disclosure, the personal care compositions may (in certain non-limiting embodiments) comprise of from 0.5% wt to 20% wt of the at least one non-sulfonate anionic surfactant. In a particular (but non-limiting) embodiment of the first aspect of the present disclosure, the compositions comprise of from 2% wt to 15% wt of the at least one non-sulfonate anionic surfactant based on the total weight of the composition.

In certain particular (but non-limiting) embodiments, the amphoteric surfactant present in the compositions of the present disclosure comprises at least one betaine surfactant represented by the general formula:

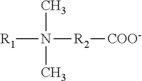

wherein: R₁ is an alkyl group containing from 8 to 18 carbon atoms, or the amido radical which may be represented by the following general formula:

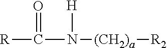

wherein: R is an alkyl group having from 8 to 18 carbon atoms, a is an integer having a value of from 1 to 4 inclusive, and R₂ is a C₁-C₄ alkylene group.

In certain particular (but non-limiting) embodiments, the at least one betaine is selected from the group consisting of dodecyl dimethyl betaine, cetyl dimethyl betaine, dodecyl amidopropyldimethyl betaine, tetradecyl dimethyl betaine, tetradecyl amidopropyldimethyl betaine, dodecyl dimethyl ammonium hexanoate and cocoamidopropyl betaine. In a particular (but non-limiting) embodiment, the at least one betaine is cocoamidopropyl betaine.

In certain particular (but non-limiting) embodiments, the compositions of the present disclosure comprise of from 0.01% wt to 10% wt of the at least one amphoteric surfactant, based on the total weight of the composition.

In certain particular (but non-limiting) embodiments, the at least two carboxylic acids or salts thereof comprise at least one monocarboxylic acid or salt thereof.

In certain particular (but non-limiting) embodiments, the least one monocarboxylic acid is selected from the group consisting of acetic acid, glycolic acid, butanoic acid, lactic acid, salicylic acid, benzoic acid and nicotinic acid. In certain particular (but non-limiting) embodiments, the at least one monocarboxylic acid comprises lactic acid.

In certain particular (but non-limiting) embodiments, the compositions of the present disclosure are substantially free of salicylic acid.

According to the second non-limiting embodiment of the first aspect of the present disclosure, the at least two carboxylic acids or salts thereof may optionally comprise at least one tricarboxylic acid or salt thereof. In certain particular (but non-limiting) embodiments, the at least one tricarboxylic acid is selected from the group consisting of citric acid, propane-1,2,3-tricarboxylic acid, aconitic acid, agaric acid and trimesic acid. In a particular (but non-limiting) embodiment, the at least one tricarboxylic acid comprises citric acid.

According to both non-limiting embodiments of the first aspect of the present disclosure, the at least two carboxylic acids or salts thereof may optionally comprise at least one monocarboxylic acid and at least one tricarboxylic acid. A particular (but non-limiting) combination of at least one monocarboxylic acid and at least one tricarboxylic acid comprises lactic acid and citric acid.

In certain particular (but non-limiting) embodiments, the at least two carboxylic acids or salts thereof are present in the compositions of the present disclosure in a total amount of from about 0.1% wt to about 10% wt based on the total weight of the composition.

In certain particular (but non-limiting) embodiments, the compositions of the present disclosure comprise at least one monocarboxylic acid or a salt thereof in an amount of from 0.1% wt. to 5% wt based on the total weight of the composition. In certain particular (but non-limiting) embodiments, the compositions of the present disclosure comprise at least one tricarboxylic acid or a salt thereof in an amount of from 0.1% wt. to 5% wt based on the total weight of the composition.

According to the first non-limiting embodiment of the first aspect of the present disclosure, the compositions may optionally comprise at least one tricarboxylic acid and at least one monocarboxylic acid in a weight ratio in the range of from 5:1 to 1:5, such as (but not limited to) of from 1:1 to 1:5. According to the second non-limiting embodiment of the first aspect of the present disclosure, the compositions may optionally comprise at least one tricarboxylic acid and at least one monocarboxylic acid in a weight ratio in the range of from 1:1.1 to 1:4.

In certain particular (but non-limiting) embodiments, the compositions of the present disclosure have a pH in the range of from about 3.3 to about 5.

In certain particular (but non-limiting) embodiments, the compositions of the present disclosure are substantially free of sulfonated compounds.

In certain particular (but non-limiting) embodiments, the compositions are substantially free of para-chloro-meta-xylenol.

In certain particular (but non-limiting) embodiments, the compositions of the present disclosure further comprise a nonionic surfactant. In certain particular (but non-limiting) embodiments, the compositions further comprise a nonionic surfactant in an amount of from 0.01% wt. to 15% wt based on the total weight of the composition.

In certain particular (but non-limiting) embodiments, the personal care compositions of the present disclosure are formulated to be showering or bathing products, hand washing products, body care products, hand care products, foot care products, facial care products, scalp care products and haircare products.

According to a second aspect of the present disclosure, there is provided a dispenser containing a composition according to the present disclosure.

According to a third aspect of the present disclosure, there is provided a disposable wipe comprising a composition of the present disclosure.

According to a fourth aspect of the present disclosure, there is provided the use of a composition according to the present disclosure to provide an antimicrobial benefit to skin and/or hair by contacting the composition with skin and/or hair.

According to a fifth aspect of the present disclosure, there is provided a method for providing an antimicrobial benefit to skin and/or hair, comprising the steps of contacting a composition according claim 1 with an area of skin and/or hair in a suitable amount and for sufficient time to provide an antimicrobial effect thereon.

The compositions of the present disclosure have been found to address one of more of the aforementioned problems described above.

In particular the inventors have found that by controlling the level of sulphonate compounds, PCMX and salicylic acid and using the carboxylic acids and non-sulfonate surfactants as described herein, personal care compositions providing effective cleansing and sanitization are provided which are well tolerated by skin and hair even upon frequent use.

Other features and advantages of the present disclosure will be apparent from the following detailed description of the present disclosure and from the accompanying claims.

The compositions according to the present disclosure will now be described in more detail.

The compositions of the present disclosure are suitably personal care compositions suitable for application to the skin and/or hair of humans, especially to human skin. In certain particular (but non-limiting) embodiments, the compositions are liquid compositions, such as (but not limited to) aqueous compositions and in particular (but not by way of limitation), liquid aqueous compositions.

The term “sulfonated compounds” means any compound containing a sulfonate group.

The term “antimicrobial” as used herein means that the control of at least one of bacteria, fungi and/or virus growth and especially of at least bacteria and viruses.

By the expression “frequent” refers to regular use of the compositions of the present disclosure. The duration between uses will depend on the type of personal care composition. For example, a handwash may be used several times a day or even several times during a couple of hours. A bodywash is more likely to be used daily, or every few days.

The amounts of ingredients stated herein refers to the amount of active ingredient based on the total weight of the composition. Unless otherwise stated, all weights are as wt % based on the total weight of the composition.

It is to be understood that all references to “% wt” amounts are to amounts which are about the stated amount. For example, a reference to ‘of from 5% wt to 10% wt’ is to be construed as ‘of from about 5% wt to about 10% wt’ unless the context requires otherwise.

### Surfactant

The compositions of the present disclosure comprise at least one anionic surfactant which does not contain a sulfonate group, and, at least one amphoteric surfactant.

### Anionic Surfactant which does not Contain a Sulphonate Group

The compositions of the present disclosure comprise at least one anionic surfactant which does not contain a sulfonate group (hereinafter non-sulfonate anionic surfactant).

In certain particular (but non-limiting) embodiments, the non-sulfonate anionic surfactant is selected from the group consisting of alkyl sulfates or salts thereof and alkyl ether sulfates or salts thereof. These sulfate surfactants provide good foaming characteristics in use and so aid cleansing of the user and also exhibit acceptable skin tolerance characteristics.

One group of particular (but non-limiting) non-sulfonate anionic surfactants are alkyl sulfates, such as (but not limited to) C₈-C₁₈ alkyl sulfates, and in particular (but not by way of limitation) C₁₀-C₁₆ alkyl sulfates such as C₁₂-C₁₄ alkyl sulfates and their salts. Any suitable salt may be used and typically an alkali metal (such as sodium, potassium, or lithium) or alkaline earth metal salt is used. In certain particular (but non-limiting) embodiments, the sodium salt is used. A particular (but non-limiting) non-sulfonate anionic surfactant is lauryl sulfate, such as (but not limited to) sodium lauryl sulfate known as SLS.

Another group of particular (but non-limiting) non-sulfonate anionic surfactants is the alkyl ether sulfates, especially C₈-C₁₈ alkyl ether sulfates, most especially C₁₀-C₁₆ alkyl ether sulfates, such as C₁₂-C₁₄ alkyl ether sulfates and their salts. Again, any suitable salt may be used and typically an alkali metal (such as sodium, potassium or lithium) or alkaline earth metal salt is used. In certain particular (but non-limiting) embodiments, the sodium salt is used. A particular (but non-limiting) non-sulfonate anionic surfactant is lauryl ether sulphate, particularly sodium lauryl ether sulphate known as SLES or sodium laureth sulfate.

Alkyl ether sulfates may be utilized according to the present disclosure as, without wishing to be bound by theory, they are believed to provide good foaming characteristics and skin tolerance in compositions with antimicrobial properties. Combinations of one or more alkyl sulfates with one or more alkyl ether sulfates may be used.

Examples of other non-sulfonate anionic surfactants which may be used according to the present disclosure include alcohol sulfates, alcohol phosphates and phosphonates, alkyl ester sulfates, sulfate esters of an alkyl phenoxy polyoxyethylene ethanol, alkyl monoglyceride sulfates, alkylaryl sulfates, alkyl carboxylates, alkyl ether carboxylates, alkyl alkoxy carboxylates having 1 to 5 moles of ethylene oxide, alkyl polyglycol ether sulfates (containing up to 10 moles of ethylene oxide), sulfosuccinates, octoxynol or nonoxynol phosphates, taurates, fatty taurides, fatty acid amide polyoxyethylene sulfates, fatty oleyl glycerol sulfates, alkyl phenol ethylene oxide ether sulfates, alkyl phosphates, isethionates, N-acyl taurates, alkyl succinamates and sulfosuccinates, alkyl polysaccharide sulfates, alkyl polyglucoside sulfates, alkyl polyethoxy carboxylates, and sarcosinates or mixtures thereof.

Still further examples of non-sulfonate anionic surfactants which may find use in the compositions of the present disclosure include alkyl-carboxylates, salts (including, for example, sodium, potassium, ammonium, and substituted ammonium salts such as mono-, di- and triethanolamine salts) of soap, C₆-C₂₄ alkyl polyglycol ether sulfates, alkyl ester sulfates such as C₁₄₋₁₆ methyl ester sulfates, fatty oleyl glycerol sulfates, alkyl phenol ethylene oxide ether sulfates, alkyl phosphates, isethionates such as the acyl isethionates, N-acyl taurates, alkyl succinamates, acyl sarcosinates, sulfates of alkyl polysaccharides such as the sulfates of alkyl polyglucoside, branched primary alkyl sulfates, alkyl polyethoxy carboxylates such as those of the formula RO(CH₂CH₂O)ₖCH₂COO⁻M⁺ wherein R is a C₈-C₂₂ alkyl, k is an integer from 0 to 10, and M is a soluble salt-forming cation.

Non-sulfonate anionic compounds which function both as surfactants and as a hydrotrope may be included as either part of the anionic surfactant or as a co-surfactant.

Exemplary hydrotropes include C₆-C₁₁ alkyl sulfates and phosphate ester hydrotropes. The hydrotropes are often provided in a salt form with a suitable counterion, such as one or more alkali, or alkali earth metals, such as sodium or potassium, especially sodium. However, other water-soluble cations such as ammonium, mono-, di-and tri-lower alkyl, i.e., C₁₋₄ alkanol ammonium groups can be used in the place of the alkali metal cations. Combinations of two or more non-sulfonate anionic surfactants may also be used in the compositions of the present disclosure if desired. However, according to one aspect of the present disclosure, it has been found beneficial for the compositions to comprise either an alkyl sulphate or an alkyl ether sulphate as the only anionic surfactant, especially an alkyl ether sulfate. In particular (but not by way of limitation), sodium lauryl sulfate or sodium lauryl ether sulphate may optionally be the only non-sulfonate anionic surfactant present in the compositions, and in particular (but not by way of limitation) sodium lauryl ether sulphate. Certain particular (but non-limiting) embodiments include the use of an alkyl sulfate and salts thereof and/or an alkyl ether sulphate and salts thereof in the amounts stated herein. A particular (but non-limiting) embodiment is the use of lauryl sulfate and/or lauryl ether sulfate, such as (but not limited to) the sodium salts thereof in the amounts stated herein.

In certain particular (but non-limiting) embodiments, the personal care compositions of the present disclosure optionally comprise of from 0.1% wt to 20% wt in total of the at least one non-sulfonate anionic surfactant, based on the total weight of the composition, such as (but not limited to) of from 0.5% wt to 15% wt, or 1% wt to 12% wt, or 2% wt to 10% wt, or 5% wt to 9% wt, based on the total weight of the composition. According to one non-limiting embodiment of the present disclosure, the compositions comprise the non-sulfonate anionic surfactant in an amount of from 0.1% wt to 20% wt based on the total weight of the composition. Particular (but non-limiting) amounts according to this embodiment are as given above.

Desirably a major proportion of the total amount of non-sulfate anionic surfactant present in the composition of the present disclosure comprises one or more alkyl sulfates and/or alkyl ether sulfates, such as (but not limited to) at least 55% wt., or at least 70% wt., or at least 80% wt., or at least 90% wt. In certain particular (but non-limiting) embodiments, the non-sulfate anionic surfactant constituent comprises at least 99% wt., of one or more alkyl ether sulfates. In a particular (but non-limiting) embodiment, the non-sulfate anionic surfactant consists essentially of one or more alkyl sulfates and/or one or more alkyl ether sulfates.

### Amphoteric Surfactants

The compositions of the present disclosure comprise at least one amphoteric surfactant.

Exemplary useful amphoteric surfactants include derivatives of secondary and tertiary amines having aliphatic radicals that are straight chain or branched, and wherein one of the aliphatic substituents contains from about 8 to 18 carbon atoms and at least one of the aliphatic substituents contains an anionic water-solubilizing group, e.g., a carboxy, sulfonate, or a sulfate group. Non-limiting examples of compounds falling within this description include: sodium 3-(dodecylamino)propionate, sodium 3-(dodecylamino)propane-1-sulfonate, sodium 2-(dodecylamino)ethyl sulfate, sodium 2-(dimethylamino)octadecanoate, disodium 3-(N carboxymethyldodecylamino)propane-1-sulfonate, disodium octadecyliminodiacetate, sodium 1-carboxymethyl-2-undecylimidazole, and sodium N,N-bis(2-hydroxyethyl)-2-sulfato-3-dodecoxypropylamine.

Further exemplary useful amphoteric surfactants include betaines including phosphobetaines. According to one non-limiting aspect of the present disclosure, betaine surfactants are optionally used as the amphoteric surfactant.

Exemplary useful betaine surfactants which may be represented by the general formula:

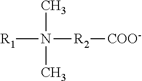

wherein: R₁ is an alkyl group containing from 8 to 18 carbon atoms, or the amido radical which may be represented by the following general formula:

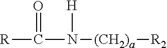

wherein: R is an alkyl group having from 8 to 18 carbon atoms, a is an integer having a value of from 1 to 4 inclusive, and R₂ is a C₁-C₄ alkylene group.

Examples of particular (but non-limiting) betaines according to the present disclosure include dodecyl dimethyl betaine, cetyl dimethyl betaine, dodecyl amidopropyldimethyl betaine, tetradecyl dimethyl betaine, tetradecyl amidopropyldimethyl betaine, dodecyl dimethyl ammonium hexanoate and cocoamidopropyl betaine. In a particular (but non-limiting) embodiment, cocoamidopropyl betaine is the betaine according to the present disclosure.

Mixtures of two or more amphoteric surfactants may be used in the compositions of the present disclosure. If a mixture is used, in certain non-limiting embodiments, the mixture comprises at least one betaine.

The at least one amphoteric surfactant, e.g. betaine, may be present in any effective amount, for example (but not by way of limitation) in amounts of from 0.01% wt. to 10% wt., or of from 0.025% wt to 7.5% wt., or of from 0.05% wt to 5% wt., or of from 0.1% wt to 2.5% wt., based on the total weight of the composition.

The compositions of the present disclosure may comprise an equal amount (% wt) of non-sulfonate anionic surfactant and amphoteric surfactant. However, in certain particular (but non-limiting) embodiments, the compositions comprise a greater % wt of non-sulfonate anionic surfactant than amphoteric surfactant. According to one non-limiting embodiment, it has been found that the weight ratio of non-sulfonate anionic surfactant to amphoteric surfactant is in the range of from 10:1 to 60:1, such as (but not limited to) 20 to 55:1, or 30:1 to 50:1, or 40:1 to 50:1. In this non-limiting embodiment, nonionic surfactant is not typically present in the compositions. According to another non-limiting embodiment, it has been found that especially good results are obtained with the compositions of the present disclosure when the weight ratio of non-sulfonate anionic surfactant to amphoteric surfactant is in the range of from 2:1 to 10:1, such as (but not limited to) 3:1 to 9:1, or 4:1 to 8:1, or 5:1 to 7:1. In this non-limiting embodiment, nonionic surfactant is also typically present in the compositions in the amounts described herein.

### Carboxylic Acids

The compositions of the present disclosure comprise at least two one carboxylic acids or salts thereof; however, the compositions of the present disclosure do not contain salicylic acid in an amount above 0.25% wt. The compositions of the present disclosure may comprise monocarboxylic acids, dicarboxylic acids and/or tricarboxylic acids and mixtures thereof. Any carboxylic acids suitable for inclusion in personal care compositions may be included in the compositions of the present disclosure. Typically, the carboxylic acids used are C₂-C₂₅ carboxylic acids, such as (but not limited to) C₃-C₂₂. According to one specific embodiment of the present disclosure, the at least two carboxylic acids or salts thereof comprises citric acid. In this embodiment, the compositions may also comprise an additional tricarboxylic acid, such as (but not limited to) one disclosed hereinbelow.

In some non-limiting embodiments of the present disclosure, the acids are utilized instead of the salts for inclusion in the personal care compositions. In some non-limiting embodiments, combinations of acids and salts may also be used if desired. The carboxylic acids or salts thereof used in the compositions of the present disclosure are those which exhibit antimicrobial properties yet are suitable for use in personal care formulations.

For the sake of brevity, the references herein to any of the carboxylic acids (generic or specific) includes reference to any salts thereof unless the context requires otherwise.

According to one non-limiting aspect of the present disclosure, the at least two carboxylic acids or salts thereof optionally comprise at least one monocarboxylic acid or salt thereof.

Any monocarboxylic acid suitable for use in personal care compositions may be used according to the present disclosure, including acetic acid, glycolic acid, butanoic acid, lactic acid, salicylic acid, benzoic acid, and nicotinic acid. If salicylic acid is used, it is not present in the compositions in an amount above 0.25% wt, such as (but not limited to) not above 0.2% wt, not above 0.15% wt, not above 0.1% wt, or not above 0.05% wt. In certain particular (but non-limiting) embodiments, the compositions of the present disclosure are substantially free from salicylic acid and most especially that they are free of salicylic acid. In certain particular (but non-limiting) embodiments, Lactic acid is utilized according to the present disclosure. According to one particular (but non-limiting) embodiment of the present disclosure, the compositions comprise less than 1% wt of salicylic acid, or less than 0.5% wt salicylic acid based on the total weight of the compositions, and in certain non-limiting embodiments, are substantially free of salicylic acid or are free of salicylic acid.

Any dicarboxylic acids suitable for use in personal care compositions may be used in the compositions of the present disclosure including oxalic, acid, succinic acid, malonic acid, tartaric acid, glutaric acid and adipic acid.

In certain particular (but non-limiting) embodiments, the at least two carboxylic acids or salts thereof comprise at least one tricarboxylic acid or salt thereof. In certain particular (but non-limiting) embodiments, the at least one tricarboxylic acid is selected from the group consisting of citric acid, propane-1,2,3-tricarboxylic acid, aconitic acid, agaric acid and trimesic acid. In certain particular (but non-limiting) embodiments, the at least one tricarboxylic acid comprises citric acid.

According to a particular (but non-limiting) aspect of the present disclosure, the at least two carboxylic acids or salts thereof optionally comprise at least one monocarboxylic acid and at least one tricarboxylic acid. A particular (but non-limiting) combination of at least one monocarboxylic acid and at least one tricarboxylic acid comprises lactic acid and citric acid.

It is also possible, according to the present disclosure to use at least one dicarboxylic acid with either at least one monocarboxylic acid and/or at least one tricarboxylic acid.

In certain particular (but non-limiting) embodiments, the at least two carboxylic acids or salts thereof are present in the compositions of the present disclosure in a total amount of from about 0.1% wt. to about 10% wt., such as (but not limited to) about 0.25% wt. to 7.5% wt., or 0.5% wt. to 5% wt., or 0.75% wt. to 4.5% wt., or 1% wt. to 3% wt., or 1.5% wt. to 2.5% wt., based on the total weight of the composition.

In certain particular (but non-limiting) embodiments, the compositions of the present disclosure comprise at least one monocarboxylic acid or a salt thereof in an amount of from about 0.1% wt. to about 5% wt., such as (but not limited to) 0.25% wt. to about 4.5% wt., or 0.5% wt. to 3.5% wt., or 0.75% wt to 3% wt., or 0.9% wt to 2.75% wt, or 1% wt. to 2.5% wt. based on the total weight of the composition. In certain particular (but non-limiting) embodiments, the compositions of the present disclosure comprise at least one tricarboxylic acid or a salt thereof in an amount of from 0.1% wt. to 5% wt., such as (but not limited to) 0.25% wt. to 4.5% wt., or 0.5% wt. to 3.5% wt., or 0.75% wt to 3% wt., or 0.9% wt to 2.75% wt, or 1% wt. to 2.5% wt. based on the total weight of the composition.

The compositions of the present disclosure may contain the at least one monocarboxylic acid or the at least one tricarboxylic acid in the aforementioned amounts. According to one aspect of the present disclosure, the compositions comprise both the at least one monocarboxylic acid and the at least one tricarboxylic acid in the aforementioned amounts.

If the compositions of the present disclosure comprise dicarboxylic acids, these acids may also be present in the amounts given above for the at least one monocarboxylic acid and the at least one tricarboxylic acid.

Particular (but non-limiting) examples of carboxylic acids and mixtures thereof according to the present disclosure, and the non-limiting exemplary amounts thereof, are disclosed in one or more of the Examples.

The inventors have observed that good results are obtained when the compositions of the present disclosure comprise at least one tricarboxylic acid and at least one monocarboxylic acid in a weight ratio in the range of from 5:1 to 1:5, such as (but not limited to) 2.5:1 to 1:5. The inventors have observed that especially good results are obtained when the compositions of the present disclosure comprise at least one tricarboxylic acid and at least one monocarboxylic acid in an equal wt % and, in a particular (but non-limiting) example, in a higher wt % of the monocarboxylic acid than of the tricarboxylic acid. Accordingly, the wt ratio of the at least one tricarboxylic acid to the at least one monocarboxylic acid is, in certain non-limiting embodiments, in the range of from 1:1 to 1:5, such as 1:1.1 to 1:4, e.g. 1:1.25 to 1:3 or 1:1.3 to 1:2.5 and most especially 1:1.4 to 1:2. In one particular (but non-limiting) embodiment of the present disclosure, the compositions comprise at least one tricarboxylic acid and at least one monocarboxylic acid in a weight ratio of 1:1.5.

In certain particular (but non-limiting) embodiments, citric acid and lactic acid are present in the aforementioned ratios.

### Sulfonated Compounds

The compositions of the present disclosure comprise no more than 30%, of the total wt % of the at least one non-sulfonate anionic surfactant and the at least one amphoteric surfactant, of sulfonated compounds. In certain particular (but non-limiting) embodiments, the compositions comprise no more than 25%, such as (but not limited to) no more than 20%, or no more than 15%, or no more than 10%, or no more than 5% of the total wt % of the at least one non-sulfonate anionic surfactant and the at least one amphoteric surfactant, of sulfonated compounds. In certain particular (but non-limiting) embodiments, the compositions of the present disclosure are substantially free of sulfonated compounds and, in particular (but non-limiting) embodiments, the compositions are free of sulfonated compounds.

The inventors have found that by controlling the content of sulfonated compounds in the compositions of the present disclosure, and by including the other components of the present disclosure, effective cleansing compositions with antimicrobial benefits can be provided without undesirable levels of harshness on the skin and/or hair being treated.

Examples of sulfonate containing compounds which may typically be included in personal care compositions include anionic sulfonate surfactants such as alcohol sulfonates, alkyl diphenyl ether sulfonates, alkyl sulfonates such as C₆-C₂₂ primary or secondary alkane sulfonates, C₆-C₂₀ linear alkylbenzene sulfonates, olefin sulfonates, beta-alkoxy alkane sulfonates, alkyl ether sulfonates, ethoxylated alkyl sulfonates, alkylaryl sulfonates, alkyl monoglyceride sulfonates, acyl glycerol sulfonates and paraffin sulfonates, sulfonated polycarboxylic acids, isethionates, N-acyl taurates, and sulfosuccinates and the monoesters and diesters thereof.

Additional anionic compounds containing a sulfonate group and functioning both as a surfactant and as a hydrotrope which may typically be used in personal care compositions include benzene sulfonates, naphthalene sulfonates, C₁-C₁₁ alkyl benzene sulfonates, xylene sulfonates, toluene sulfonates, cumene sulfonates, C₅-C₁₁ alkyl sulfonates and alkyl diphenyloxide disulfonate and their salts.

The amounts of such sulfonated compounds are controlled in the compositions of the present disclosure. Such control can lead, for example, to improved skin and hair tolerance of the compositions of the present disclosure compared to similar compositions containing higher levels of sulfonated compounds.

According to a first embodiment of the first aspect of the present disclosure, there is therefore provided personal care compositions comprising:
a) 0.1% wt to 20% wt of at least one non-sulfonate anionic surfactant,
b) at least one amphoteric surfactant,
c) at least two carboxylic acids or salts thereof, and
   wherein the at least two carboxylic acids or salts thereof comprises citric acid and the composition contains no more than 30% of the total wt % of a) and b) of sulfonated compounds, and, the compositions do not contain para-chloro-meta-xylenol in an amount above 0.15% wt and do not contain salicylic acid in an amount above 0.25% wt.

A particular (but non-limiting) composition according to the first embodiment of the first aspect of the present disclosure comprises:
a) 0.1% wt to 20% wt of at least one non-sulfonate anionic surfactant comprising alkyl ether sulfate,
b) at least one amphoteric surfactant comprising a betaine,
c) at least two carboxylic acids or salts thereof, and
   wherein the at least two carboxylic acids or salts thereof comprises citric acid and lactic acid and wherein the compositions contain no more than 30% of the total wt % of a) and b) of sulfonated compounds, and, further wherein the compositions do not contain para-chloro-meta-xylenol in an amount above 0.15% wt and do not contain salicylic acid in an amount above 0.25% wt.

According to a second embodiment of the first aspect of the present disclosure, there is therefore provided personal care compositions comprising:
a) at least one non-sulfonate anionic surfactant,
b) at least one amphoteric surfactant,
c) at least two carboxylic acids or salts having different number of carboxylic acid groups, and
   wherein the compositions comprise a higher % wt of the carboxylic acid having the lowest number of carboxylic acid groups, and no more than 30% of the total wt % of a) and b) of sulfonated compounds, and, the compositions do not contain para-chloro-meta-xylenol in an amount above 0.15% wt and do not contain salicylic acid in an amount above 0.25% wt.

A particular (but non-limiting) composition according to the second embodiment of the first aspect of the present disclosure comprises:
a) at least one non-sulfonate anionic surfactant comprising alkyl ether sulfate,
b) at least one amphoteric surfactant comprising a betaine,
c) at least two carboxylic acids or salts having different number of carboxylic acid groups and comprising lactic acid and citric acid, and
   and wherein the compositions comprise a higher % wt of the lactic acid, and no more than 30% of the total wt % of a) and b) of sulfonated compounds, and, further wherein the compositions do not contain para-chloro-meta-xylenol in an amount above 0.15% wt and do not contain salicylic acid in an amount above 0.25% wt.

### Optional Ingredients

The compositions of the present disclosure may include one or more further optional constituents which are used to impart one or more desired esthetic or technical benefits to the compositions. Such optional ingredients include additives and adjuvants which are conventional in the personal care field, such as moisturisers, skin conditioning agents, fragrances, essential oils, colorants, preservatives, further antimicrobially active compounds or materials, foam boosters, humectants, opacifiers, antioxidants, chelating agents, thickener and light stabilizers including UV absorbers. The amounts of these various additives and adjuvants are those conventionally used in the field.

In certain particular (but non-limiting) embodiments, the compositions of the present disclosure are liquid in form. In certain particular (but non-limiting) embodiments, they are aqueous based compositions. Particular (but non-limiting) embodiments are liquid, aqueous based, compositions. Water may form the major component of the compositions of the present disclosure. The compositions typically comprise water in an amount in the range of from 65% wt to 99% wt, based on the total weight of the composition. In certain particular (but non-limiting) embodiments, the compositions comprise an amount of from 70% wt to 98% wt water, such as (but not limited to) of from 75% wt to 95% wt, or in the range of from 80% wt to 90 wt %.

The water may be tap water, but in certain particular (but non-limiting) embodiments is distilled water or deionized water or “soft” water. If the water is tap water, in certain particular (but non-limiting) embodiments, it is substantially free of any undesirable impurities such as organics or inorganics, especially minerals salts which are present in hard water which may undesirably interfere with the operation of the ingredients present in the compositions according to the present disclosure.

The compositions of the present disclosure may include one or more chelating agents. Exemplary useful chelating agents include those known to the art, including by way of non-limiting example; aminopolycarboxylic acids and salts thereof wherein the amino nitrogen has attached thereto two or more substituent groups. Particular (but non-limiting) chelating agents include acids and salts, especially the sodium and potassium salts of ethylenediaminetetraacetic acid, diethylenetriaminepentaacetic acid, N-hydroxyethylethylenediaminetriacetic acid, and of which the sodium salts of ethylenediaminetetraacetic acid may be particularly advantageously used. If present, chelating agents are typically included in a total amount in the range of from 0.01 to 0.5% wt., especially of from 0.01 to 0.25% wt based on the total weight of the composition.

The compositions of the present disclosure may comprise one or more humectants, for example polyhydric alcohols including polyalkylene glycols as well as alkylene polyols and their derivatives, such as propylene glycol, dipropylene glycol, polypropylene glycol, polyethylene glycol and derivatives thereof, sorbitol, hydroxypropyl sorbitol, erythritol, threitol, pentaerythritol, xylitol, glucitol, mannitol, hexylene glycol, butylene glycol (e.g., 1,3-butylene glycol), hexane triol (e.g., 1,2,6-hexanetriol), glycerine, ethoxylated glycerine and propoxylated glycerine. Further useful humectants include sodium 2-pyrrolidone-5-carboxylate, guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; and, panthenol. In certain particular (but non-limiting) embodiments, Glycerine is the humectant according to the present disclosure. One or more humectants may be included in the compositions of the present disclosure. Aloe vera, in one or more of its forms, is a particular (but non-limiting) humectant, as being a naturally derived product. When present, one or more humectants may be included in effective total amounts, advantageously from 0.01-5% wt., such as (but not limited to) from 0.01-3% wt., or from 0.5% wt to 2% wt based on the total weight of the composition of which it/they form(s) a part.

The compositions of the present disclosure may, if desired, include suitable pH adjusting agents including phosphorus containing compounds, monovalent and polyvalent salts such as of silicates, carbonates, and borates, certain acids and bases, tartrates and certain acetates. In certain particular (but non-limiting) embodiments, a base as a pH adjuster, such as a hydroxide, for example an alkali metal or alkaline earth metal hydroxide, is utilized according to the present disclosure. In certain particular (but non-limiting) embodiments, sodium hydroxide is used as a pH adjustment agent in the compositions of the present disclosure.

Other pH buffering compounds which may also be used in the compositions include the alkali metal phosphates, polyphosphates, pyrophosphates, triphosphates, tetraphosphates, silicates, metasilicates, polysilicates, carbonates, hydroxides, and mixtures of the same. Certain salts, such as the alkaline earth phosphates, carbonates, hydroxides, can also function as buffers. It may also be desirable to use as buffers such materials as aluminosilicates (zeolites), borates, aluminates and certain organic materials such as gluconates, succinates, maleates, and their alkali metal salts.

When present, the pH adjusting agent is present in an amount effective to adjust or maintain the pH of the inventive composition within a target pH range. Typically, the pH adjusting agent may be included in relatively minor amounts such as from 0.01-5% wt. dependent on the degree of pH adjustment required to achieve the desired pH. If included, they are desirably present in amounts from 0.1-2.5% wt, such as 1-2% wt.

In certain particular (but non-limiting) embodiments, the compositions of the present disclosure comprise at least one preservative. Preservatives are added to inhibit the growth of bacteria, fungi and/or yeasts and many suitable preservatives are known in the art. In certain non-limiting embodiments of the compositions of the present disclosure, a preservative which is effective against fungi and/or moulds is optionally included therein. Exemplary useful preservatives include benzoates, such as sodium benzoate. Parabens may also be used in place of benzoates. Advantageously one or more preservatives may be included in amounts of about 0.05% wt. to about 2% by weight, based on the total weight of the composition of which the preservative forms a part. When present, the preservative is included in any amount found to be effective in retarding or inhibiting the growth of undesired microorganisms in the compositions of the present disclosure, particularly during storage for several months at room temperature. The preservative is advantageously present in amounts of up to about 2% wt., such as (but not by way of limitation) in amounts of from about 0.001% wt. to about 1.5% wt., or is present in an amount of from about 0.01% wt. to 1.25% wt, such as from about 0.05% wt. to 1% wt based on the total weight of the composition of which it forms a part.

The compositions of the present disclosure may also include a material which is able to boost the effect of the preservative. These materials are herein termed “preservative boosters”. Such preservative boosters predominantly exhibit other characteristics as well. They typically exhibit some degree of antimicrobial effect although not usually sufficient to be used alone to provide either the preservative effect, or, the antimicrobial effect required for the compositions. Alcohols, such as glycols, may exhibit this preservative booster effect, especially alkyl glycols, e.g. C₂-C₁₀ glycols. One such non-limiting example is caprylyl glycol, which is an optional ingredient of the compositions of the present disclosure. Caprylyl glycol's main function is to provide emolliency properties to the compositions, but, as it has a degree of antimicrobial activity, it also assists the preservative and provides a boost in preservative power to the compositions. A suitable commercially available caprylyl glycol product is Microcare® CLG (ex Thor). Other suitable preservative boosters include benzyl alcohol. The preservative booster is advantageously present in amounts of up to about 1% wt., such as (but not limited to) is present in amounts of from about 0.001% wt. to about 0.5% wt., or is present in an amount of from about 0.01% wt. to 0.25% wt, such as from about 0.01% wt. to 0.2% wt based on the total weight of the composition of which it forms a part.

The topical compositions may include a fragrance, which may be based on natural and/or synthetic fragrances and most commonly are mixtures or blends of a plurality of such fragrances, optionally in conjunction with a carrier such as an organic solvent or a mixture of organic solvents in which the fragrances are dissolved, suspended or dispersed. When present the fragrance may be present in any effective amount such that it can be discerned by a user of the composition. However, it is advantageously present in amounts of up to about 1% wt., or is present in amounts of from about 0.0001% wt. to about 0.5% wt based on the total weight of the composition of which it forms a part.

The inventive compositions may include one or more colorants, e.g., dyes or pigments useful in personal care compositions to impart a desired color or tint to the compositions. Any colorant which is compatible with the other constituents in the compositions of the present disclosure may be included and in any suitable amount required to achieve the desired visual effect. Advantageously one or more colorants may be added in amounts of about 0.001% wt. to about 0.5% by weight, based on the total weight of the composition of which the colorant(s) forms a part.

If a pearlescent appearance is desired for the compositions of the present disclosure, any suitable pearlizing agents may be added in conventional amounts. A particular (but non-limiting) type of pearlizing agent is the glycol stearates, such as ethylene glycol monostearate (EGMS). When present, the pearlizer is typically included in amounts of from about from 0.001 to 2% wt., such as (but not limited to) in amounts from 0.01 to 1.5 wt., e.g. of from about 0.05 to 1% wt. based on the total weight of the composition of which it forms a part.

Another optional ingredient which may be included in the compositions of the present disclosure is an opacifier, e.g. a latex, to provide opacification of the composition. Such are materials which are typically emulsions, dispersions or suspensions of a water insoluble polymer or copolymer in a carrier. Any suitable commercial opacifier may be used if an opaque composition is desired, such as those available under the trademark ACUSOL (ex. Rohm & Haas Inc.). When present in a composition according to the present disclosure, the opacifier may be present in amounts of up to about 5% wt., such as (but not limited to) are present in amounts of from about 0.001% wt. to about 3% wt., or are present in amount from about 0.1% wt. to about 1.2% wt, or are present in amounts of from about 0.1% wt. to about 1% wt., based on the total weight of the composition of which it forms a part.

The compositions of the present disclosure may comprise one or more nonionic surfactants. According to one non-limiting embodiment of the present disclosure, a nonionic surfactant may optionally be included in the compositions of the present disclosure. Suitable nonionic surfactants include ethoxylated alkylphenols, ethoxylated and propoxylated fatty alcohols, polyethylene glycol ethers of methyl glucose, polyethylene glycol ethers of sorbitol, ethylene oxide, propylene oxide block copolymers, ethoxylated esters of fatty (C₆-C₂₄) acids, condensation products of ethylene oxide with long chain amines or amides, and mixtures thereof. Further exemplary nonionic surfactants include, but are not limited to: methyl gluceth-10, PEG-20 methyl glucose distearate, PEG-20 methyl glucose sesquistearate, C₁₁-C₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethyiene-20 isohexadecyl ether, polyoxyethylene-23 glycerol laurate, polyoxyethylene-20 glyceryl stearate, PPG-10 methyl glucose ether, PPG-20 methyl glucose ether, polyoxyethylene-20 sorbitan monoesters, polyoxyethylene 80 castor oil, polyoxyethylene-15 tridecyl ether, polyoxyethylene-6 tridecyl ether, laureth-2, laureth-3, laureth-4, PEG-3 castor oil, PEG 600 dioleate, PEG 400 dioleate, and mixtures thereof. Alkyl polyglucosides are an especially particular (but non-limiting) type of nonionic surfactants according to the present disclosure. Any suitable alkyl polyglucoside may be used. However, C₆-C₁₈ alkyl glucosides are particular (but non-limiting) examples, for example C₈-C₁₆ alkyl glucosides such as C₁₀ glucoside. This C₁₀ glucoside is available as Planteren™ 2000 ex Cognis. Other nonionic surfactants, although not specifically disclosed herein but known to the art may also be used.

If present, the nonionic surfactant(s) may optionally be included in a total amount of from about 0.01% wt. to about 15% wt., such as (but not limited to) of from 0.1% wt. to 10% wt., or 0.5% wt to 7.5% wt., or 1% wt to 5% wt, or 1.5% wt to 3% wt., based on the total weight of the composition of which they form a part.

The inventors have also found personal care compositions comprising nonionic surfactants as below to be surprisingly beneficial. The personal care compositions comprise:
a) at least one non-sulfonate anionic surfactant,
b) at least one amphoteric surfactant,
c) at least two carboxylic acids or salts,
d) at least one nonionic surfactant, and
   wherein the compositions comprise no more than 30% of the total wt % of a) and b) of sulfonated compounds.

A foam booster (which improves the foaming characteristics of the surfactant(s), especially anionic surfactant, present in the compositions of the present disclosure) may also be included. Such foam boosters are typically nonionic surfactants. Particular (but non-limiting) foam boosters are based on one or more alkanolamides which provide composition thickening, foam enhancement, and foam stability. Exemplary alkanolamides which provide such a foam boosting function include but are not limited to: cocamide MEA (monoethamolamine), cocamide DEA (diethamolamine), soyamide DEA, lauramide DEA, oleamide MIPA (monoisopropanolamide), stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA, and mixtures thereof. When present, the one or more alkanolamides are present in amounts of up to about 10% wt., but, are particularly (but not by way of limitation) included in amounts of from about 0.1-10% wt, such as 0.2-5% wt, for example 0.5-2.5% wt based on the total weight of the topical composition of which they form a part.

Where the compositions require a viscosity modifier, any suitable modifier in an appropriate amount to achieve the desired degree of viscosity modification may be used. For example, sodium chloride may be used to thicken the compositions of the present disclosure. Typically, the sodium chloride is added in the form of a solution in a concentration and an amount suitable to achieve the desired degree of thickening. Typically, the viscosity adjuster, if present, is used in amounts of 1% wt or less, such as (but not limited to) in the range of from 0.01% wt to 0.75% wt, or 0.05% wt to 0.5% wt, such as 0.1% wt to 0.4% wt.

The compositions of the present disclosure may optionally comprise further antimicrobially active materials which are effective against gram-negative and/or gram-positive bacteria provided they are compatible with the other constituents present in the composition. Exemplary useful materials which may be used as the further antimicrobially active material include one or more of antimicrobial agents including: pyrithiones, (especially zinc pyrithione which is also known as ZPT), dimethyldimethylol hydantoin (Glydant®), methylchloroisothiazolinone/methylisothiazolinone (Kathon CG®), sodium sulfite, sodium bisulfite, zinc sulfate, imidazolidinyl urea (Germall 115®), diazolidinyl urea (Germaill II®), benzyl alcohol, 2-bromo-2-nitropropane-1,3-diol (Bronopol®), formalin (formaldehyde), iodopropenyl butylcarbamate (Polyphase P100®), chloroacetamide, methanamine, methyldibromonitrile glutaronitrile (1,2-Dibromo-2,4-dicyanobutane or Tektamer®), glutaraldehyde, 5-bromo-5-nitro-1,3-dioxane (Bronidox®), phenethyl alcohol, o-phenylphenol/sodium o-phenylphenol, sodium hydroxymethylglycinate (Suttocide A®), polymethoxy bicyclic oxazolidine (Nuosept C®), dimethoxane, thimersal dichlorobenzyl alcohol, captan, chlorphenenesin, dichlorophene, chlorbutanol, glyceryl laurate, halogenated diphenyl ethers for example 2,4,4′-trichloro-2′-hydroxy-diphenyl ether (Triclosan® or TCS), phenolic compounds like phenol and aromatic halophenols, 2-hydroxydiphenylmethane, resorcinol and its derivatives, bisphenolic compounds, benzoic esters (parabens) and halogenated carbanilides. Para-chloro-meta-xylenol (PCMX) may optionally be used in the compositions of the present disclosure according to some aspects of the present disclosure, to provide an antimicrobial effect in addition to the carboxylic acids and the amphoteric surfactants. The compositions of the present disclosure do not contain para-chloro-meta-xylenol in an amount of above 0.15% wt. If used, PCMX is present in an amount of at most 0.15% wt, such as (but not limited to) less than 0.1% wt, or less than 0.05% wt based on the total weight of the compositions. In certain particular (but non-limiting) embodiments, the compositions of the present disclosure are optionally substantially free of PCMX and, in particular (but not by way of limitation), free of PCMX.

The one or more one further antimicrobially active materials may be present in conventional amounts typically of from about from 0.001-3% wt., such as (but not limited to) in amounts from 0.1-2% wt., but are most desirably present from about 0.1-0.5% wt. based on the total weight of the composition of which they form a part.

The compositions of the present disclosure may include one or more cationic Polyquaternium-type polymers if it is desired to impart moisturizing or conditioning properties to the compositions of the present disclosure and if compatible with the surfactant system used. Such materials may also have mild antimicrobial effects dependent upon the Polyquaternium polymer used. Such materials are well known to the art of topical compositions.

The one or more cationic Polyquaternium-type polymers may be present in amounts of from about from 0.001 to 2.5% wt., such as (but not limited to) in amounts from 0.01 to 2% wt., but are most desirably present in weight percentages from about 0.05 to 1% wt. based on the total weight of the composition of which they form a part.

The compositions may include one or more antioxidant constituents if desired. Examples of antioxidants include but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, glutathione, and ascorbic acid and ascorbic acid derivatives (e.g., ascorbyl palmitate and ascorbyl polypeptide). Oil-soluble antioxidants suitable for use in the compositions of this present disclosure include, but are not limited to, butylated hydroxytoluene, retinoids, tocopherols e.g., tocopherol acetate, tocotrienols, and ubiquinone. Natural extracts containing antioxidants suitable for use in the topical compositions of this present disclosure, include but not limited to, extracts containing flavonoids and isoflavonoids and their derivatives, extracts containing resveratrol and the like. Examples of such natural extracts include grape seed, green tea, pine bark and propolis. When present the total amount of such antioxidants are usually not in excess of 5% wt, such as (but not limited to) are present in amounts of from 0.0001-4% wt. based on the total weight of the composition of which it forms a part.

The compositions may comprise a thickener, if required, to achieve the desired viscosity for the compositions. Typically a polysaccharide based thickener may be included e.g., cellulose, alkyl celluloses, alkoxy celluloses, hydroxy alkyl celluloses, alkyl hydroxy alkyl celluloses, carboxy alkyl celluloses, carboxy alkyl hydroxy alkyl celluloses, and derivatives thereof including methyl cellulose ethyl cellulose, hydroxymethyl cellulose, hydroxy ethyl cellulose, hydroxy propyl cellulose, carboxy methyl cellulose, carboxy methyl hydroxyethyl cellulose, hydroxypropyl cellulose, hydroxy propyl methyl cellulose, ethylhydroxymethyl cellulose and ethyl hydroxy ethyl cellulose. Also suitable are naturally occurring polysaccharide polymers such as xanthan gum, guar gum, locust bean gum, tragacanth gum, or derivatives thereof, polycarboxylate polymers, polyacrylamides, clays, and mixtures thereof.

The polysaccharide-based thickener constituent, particularly the cellulose-based thickener constituent, may be present in any effective amount, and are present in, for example but not by way of limitation, amounts of from 0.01% wt. to 7.5% wt. based on the total weight of the composition.

The compositions of the present disclosure may include one or more essential oils which are selected to provide a so-called “aromatherapy benefit” for the user. Essential oils are well known in personal care products. The compositions of the present disclosure may comprise one or more essential oils in conventional amounts. It is also to be recognized that when used, an essential oil or oils providing an aromatherapy benefit may replace all or part of any further fragrance constituent in the compositions of the present disclosure as many of the essential oils providing an aromatherapy benefit are odiferous. These one or more essential oils may be used in the compositions of the present disclosure in an amount of about 0.00001 wt. % to about 1 wt. %, based on the total weight of the composition.

The compositions may include one or more light stabilizers as well as UV absorbers. Such materials are known to be useful in personal care compositions and impart a degree of stability to the compositions which comprise one or more components which are typically deleteriously affected when exposed to certain sources of light, e.g., sunlight, fluorescent light sources. Other such materials are known to stabilize or improve the effect of colorants which may be present in the compositions. Any cosmetically acceptable material or compound which provides protection for one or more of the constituents in the inventive compositions from photolytic degradation or photo-oxidative degradation may be used. When present, the one or more light stabilizers as well as UV absorbers may be included in any effective amount; advantageously such materials are present in amounts of from 0.0001-1% wt., such as (but not limited to) from 0.001-0.25% wt. based on the total weight of the composition of which it forms a part.

Plant or flower extracts, oils or essences may also be included in the compositions of the present disclosure, to provide desirable benefits such as additional antimicrobial properties (for example tea tree oil), moisturizing properties, soothing properties, conditioning properties and/or fragrance. Suitable extracts are well known to the formulator of personal care products. If present such extracts are included in conventional amounts and are usually not in excess of 2% wt, such as (but not limited to) are present in amounts of from 0.0001-1.5% wt, such as 0.01 to 1% wt, based on the total weight of the topical composition of which they form a part.

The compositions may comprise one or more aliphatic alcohols, such as ethanol or propanol (including propan-1-ol and propan-2-ol) if desired. If present, the alcohol is optionally present in an amount of 0.1 wt % to 20 wt %, such as (but not limited to) 0.5% wt% to 10 wt %, or 1wt % to 5wt %. In other non-limiting embodiments of the present disclosure, the compositions comprise less than 5wt % total alcohol, such as (but not limited to) less than 2.5 wt %, and may optionally be substantially free of alcohol and in particular (but not by way of limitation) are free of alcohol. The inclusion of high level of alcohols typically renders the compositions harsher to the human skin and hair meaning they are less well tolerated. Additionally, compositions comprising high levels of alcohol may require ‘flammable’ warning information on their packaging. Both these aspects are undesirable, and the present disclosure therefore seeks to provide compositions which, if they contain alcohol, do so at levels which do not undesirably damage human skin and/or hair with frequent use. Also, the present disclosure seeks to provide personal care compositions which do not need to carry ‘flammable’ warnings.

In some compositions according to the present disclosure it may be possible to include cationic surfactants. However, they are usually omitted to avoid the undesirable formation of complexes with the anionic surfactants present. In certain particular (but non-limiting) embodiments, the compositions of the present disclosure are substantially free from cationic surfactants and especially that they are free from cationic surfactants.

### pH of the Compositions

The inventors have also found that excellent antimicrobial efficacy and good human skin and/or hair tolerance is provided when the compositions of the present disclosure are formulated to be within a specific acidic pH range of from about 3.3 to about 5, and this range is desired (but not by way of limitation) according to the present disclosure. In certain particular (but non-limiting) embodiments, the compositions have a pH in the range of from about 3.5 to about 4.8, such as (but not by way of limitation) of from about 3.75 to about 4.7, or of from about 3.9 to about 4.6, or of from about 4.0 to about 4.4. It is necessary to balance antimicrobial activity and skin tolerance. Hence very low pH's (especially below pH 3) are avoided according to the present disclosure, as they are believed to be more likely to adversely affect the skin/hair potentially leading to skin irritation, sensitization, dryness and/or damage. To control the pH of the compositions, it may be necessary to add a pH adjusting agent to bring it within the desired range.

Immediately upon preparation the compositions of the present disclosure are typically acidic in nature and may require an alkaline pH adjusting agent to bring the pH into the desired range. Suitable pH adjusting agents are discussed hereinbelow.

The pH of the compositions of the present disclosure are those measured at 20° C.

When it is desirable to adjust the pH of the compositions of the present disclosure (e.g. to provide a pH which is better tolerated by skin/air than the starting pH of the composition prior to pH adjustment), one or more pH adjusting agents and/or one or more pH buffers may be included in the compositions in effective amounts to provide the desired pH as described hereinbelow.

### Format of the COMPOSITIONS of the PRESENT DISCLOSURE

In certain particular (but non-limiting) embodiments, the personal care compositions of the present disclosure are liquids. In certain particular (but non-limiting) embodiments, the compositions are clear, transparent, or translucent liquid compositions. If it desired to make the composition opaque or pearlized in appearance a suitable opacifier or pearliser may be added. If a colored composition is desired one or more suitable colorants may be added. Suitable such ingredients are described hereinabove.

The personal care compositions of the present disclosure are intended to provide antimicrobial benefits upon use and thus will be formulated in a physical form suitable for this purpose.

Liquid compositions of the present disclosure can, if desired, be formulated to be in the form of an aqueous solution, a lotion, a cream, an emulsion (including a microemulsion), a mousse or a gel as required. Solid compositions can also be provided according the present disclosure, for example a bar soap.

In certain particular (but non-limiting) embodiments, the compositions of the present disclosure produce foam when used. Foam present during use is often perceived by the user as a visual and/or tactile cue of the efficacy/activity of a composition. It also provides the advantage for the user that the coverage of the composition over the area to be treated can be visually assessed which aids the effectiveness of sanitization and reduces the likelihood of areas not being treated.

The liquid compositions of the present disclosure may exhibit a viscosity around that of water (8.9×10⁻⁴ Pa·s at about 25° C.), and for some applications, this viscosity may be desired (albeit not by way of limitation). It is also possible to formulate the compositions to have a higher viscosity (e.g. by the inclusion of thickeners) as desired. For some non-limiting embodiments of the present disclosure, personal care compositions have a viscosity in the range of from 3,000 to 15,000 Centipoise (cP) at 20° C., such as (but not limited to) in the range of from 3,500 to 13,000 cP, or of from 4,000 to 12,500 cP. The viscosity may be in the range of from 3,000 to 9,000 cP, such as (but not by way of limitation) 3,500 to 8,500 cP, or 4,000 to 8,000 cP, or 4,500 to 7,500 cP, or 4,750 to 7,000 cP. Such viscosities have been found to be especially useful as liquid handwash compositions. The viscosity may alternatively be in the range of from 7,000 to 13,000 cP, such as (but not limited to) 7,500 to 12,500 cP, or 8,000 to 12,000 cP, or 8,500 to 11,500 cP, or 9,000 to 11,000 cP. Such viscosities have been found to be especially useful as liquid bodywash compositions.

When the compositions of the present disclosure are formulated as liquid personal care products, they may be utilized in a variety of applications, for example as body washes, bathing products such as bath additives, intimate wash products, facial cleansers, liquid hand soaps, foot washes, scalp treatments or shampoos, lotions or creams for the face, hands, feet or body. Solid personal care products, such as bar soaps, cleansing bars and solid hair treatment compositions are also envisaged by the present disclosure. The compositions of the present disclosure are not intended as treatment agents for the eyes or buccal cavity.

The compositions of the present disclosure may be provided in the form of a concentrated composition which is diluted prior to use to form a composition according to the present disclosure. This provides environmental benefits as it avoids the unnecessary transportation of dilute compositions.

The compositions of the present disclosure are intended for application to the skin and/or hair to provide an antimicrobial benefit thereto. Thus, typically the compositions are provided in a dispenser, or held in, or on, a substrate (such as a non-woven material) for use in such a treatment.

The composition can be packaged in any suitable dispenser to suit its viscosity and intended use by the consumer. Suitable dispensers for the compositions of the present disclosure include bottles, spray dispensers, pump dispensers, aerosol containers, non-deformable dispensers and squeezable dispensers (where pressure is applied to the body of the dispenser, typically manual pressure) to achieve the egress of the composition from the dispenser. Suitable such dispensers are well known in the art.

Suitable substrates in, or on, which the compositions of the present disclosure may be held include wipes which are impregnated (or otherwise carry) a composition of the present disclosure. These wipes may be intended for either single or multiple use and formulated accordingly. Such wipes may be produced from non-woven substrate materials which are well known in the art and include those based on viscose, cotton, cellulose or cellulose derived materials. These items are typically referred to as ‘disposable wipes’. The substrate material is typically soaked in, impregnated with or sprayed with the composition to be delivered by use of the wipe. Such wipes can be easily carried by the user for use outside of the home when required to allow for cleansing when required.

Thus, a second embodiment of the present disclosure provides a dispenser, containing a composition according to the present disclosure.

Thus, a third embodiment of the present disclosure provides a disposable wipe comprising a composition of the present disclosure.

### Use of the Compositions of the Present Disclosure

Topical application of the compositions of the present disclosure may be performed by contacting the compositions with the skin on any part of the human body, including on the face, neck, chest, back, arms, axilla, hands, legs, feet and scalp. It may also be applied to hair if it is desired to impart an antimicrobial effect to hair. However, primarily, the topical compositions of the present disclosure are intended for application to the skin.

In one aspect of the present disclosure, use of the compositions of the present disclosure provides a rapid, effective and convenient method of providing beneficial cleansing and/or antimicrobial effects to the area being treated whilst being well tolerated by skin and hair. Where appropriate, the compositions of the present disclosure are intended to be washed off with water after use e.g. when formulated as a showering or bathing product, or as another type of washing product.

To use a composition of the present disclosure, the user may simply dispense the desired quantity of the compositions onto the part of the body to be cleansed (e.g. the hands) and rub the composition around that body part. Alternatively, the composition may be applied to a substrate and the substrate contacted with the user's skin and/or hair. Typically, the compositions of the present disclosure are rinsed off after use.

Contact times between the compositions of the present disclosure and the area of skin or hair to be treated to allow for optimal antimicrobial effects is, for example (but not by way of limitation), at least 10 seconds, or at least 15 seconds.

In certain particular (but non-limiting) embodiments, the personal care compositions of the present disclosure are formulated to be showering or bathing products (such as body washes, shower gels and products to be added to bath water), hand washing products, body care products, hand care products, foot care products, facial care products, scalp care products, or haircare products.

Thus, in a fourth embodiment, the present disclosure provides the use of compositions according to the present disclosure to provide an antimicrobial benefit to skin and/or hair by contacting the composition with skin and/or hair.

Thus, in a fifth embodiment, the present disclosure provides a method for providing an antimicrobial benefit to skin and/or hair, comprising the steps of contacting compositions according to the present disclosure with an area of skin and/or hair in a suitable amount and for sufficient time to provide an antimicrobial effect thereon.

### Antimicrobial Activity of the Compositions

In certain particular (but non-limiting) embodiments, the compositions of the present disclosure provide effective antimicrobial activity (germ killing properties) upon both gram-positive bacteria, such as Staphylococcus aureus (S. aureus), and gram-negative bacteria, such as Escherichia coli (E. coli). The antimicrobial action of the compositions of the present disclosure has been tested using the specified bacterial strains shown in the Examples, but other suitable strains could also be employed to demonstrate the antimicrobial effectiveness of the compositions of the present disclosure.

The inventors have observed that tricarboxylic acids, especially citric acid, are very effective upon gram-negative bacteria (such as E. coli), but, slightly less effective against gram-positive bacteria (such as S. aureus). The inventors have also observed that monocarboxylic acids, especially lactic acid, are effective against gram-positive bacteria (such as S. aureus) and help to provide a good level of antimicrobial activity for the composition against both gram-negative bacteria and gram-positive bacteria when used with a tricarboxylic acid.

For the antimicrobial testing results given in the Examples, while there are no established criteria for an absolute “pass” or “fail” result in such tests, it was considered by the inventors of the present compositions that a log₁₀ reduction result of less than 3 constituted a “fail” score, and a log₁₀ reduction result of 3 or more constituted a “pass” score and demonstrated superior performance against the stated gram-positive and gram-negative bacteria. In particular (but not by way of limitation), the inventors found that compositions which achieved scores of 3.5 or more, especially 4 or more, most especially 4.5 or more were desired (but not by way of limitation), as they exhibited greater antimicrobial effects as the score increased. Particular (but non-limiting) compositions of the present disclosure achieved a log₁₀ reduction score of 5 or more.

The compositions of the present disclosure are also believed to be effective against spores and certain viruses, including enveloped DNA and RNA viruses, such as those which cause the common cold, influenza viruses and coronaviruses.

### Preparation of the Compositions

The compositions of the present disclosure may be produced by any suitable method. One suitable method which may be used to produce the compositions is given below and was used to produce the examples. For any types of ingredients not included in the compositions of the present disclosure, the addition step for ingredient would simply be omitted. The preparation is typically carried out in a suitably sized mixing tank, equipped with a stirrer. Other suitable methods of preparation will be apparent to the person skilled in the art.

Add approximately 40% of the total amount of distilled water in the composition at ambient temperature and stir at a sufficient speed to produce a vortex.

Heat the water to about 65-70° C.

If using a chelating agent, such as tetrasodium EDTA, add and mix until dissolved.

Add the one or more tricarboxylic acid(s), such as citric acid (may be added as a solution if desired) and mix until dissolved.

Add the non-sulfonate anionic surfactant, such as sodium lauryl ether sulphate, and mix until fully dissolved. (e.g. for about 15 minutes).

Add the amphoteric surfactant, such as cocoamidopropyl betaine, and mix until dissolved, maintaining a temperature not above 70° C. A sample may be taken from the mixing tank to check for undissolved non-sulfonate anionic surfactant and/or amphoteric surfactant. If any is found, continue mixing until none is found.

If using a preservative system aid, such as caprylyl glycol, add the required amount (pre-heated to a temperature of approximately 35° C. to 40° C. if required to aid addition) and mix until fully dispersed.

If using a pearlizer, such as ethylene glycol monostearate, add to the mixture and mix until dissolved, ideally maintaining the temperature of the mixture in the range of about to about 65-70° C. Once the pearlizer has dissolved stop heating.

Add approximately 30% of the total amount of distilled water in the composition to the mixture and continue mixing until the mixture is well mixed.

If using a humectant, such as glycerin, add as the mixture is cooling (e.g. when the mixture is in the temperature of about 50-55° C.) and mix until dissolved.

Add the one or more monocarboxylic acid(s), such as lactic acid (may be added as a solution if desired) and mix until dissolved.

Cool the mixture to a temperature of between ambient and about 40° C., e.g. 35-40° C.

Add the required amount of preservative (e.g. sodium benzoate) and mix until fully dissolved.

If dye is used, separately dissolve it in a sufficient amount of distilled water and add the dye/water mixture to the mixing tank, mix until dissolved.

If fragrance is used, add the required amount and mix until dissolved.

Measure the pH of the composition (typically will be around pH 3-4) and adjust to a pH within the target range for the composition, typically by using a suitable base (e.g. a 30% wt solution of sodium hydroxide), noting the amount used to achieve the desired pH. Continue mixing the composition until well mixed.

Measure the viscosity of the composition and adjust as required. If desired, the viscosity can be adjusted by the addition of a viscosity adjuster such as sodium chloride (which may be used as a solution) noting the amount used to achieve the desired viscosity. Continue mixing until well mixed.

Add an amount of distilled water sufficient to make the composition up to 100 parts (water to 100%, or q.s.).

N.B. The raw material vessels, especially those containing the non-sulfonate anionic surfactant, amphoteric surfactant, carboxylic acids and any perfume may desirably be rinsed with around 1% wt of the total amount of distilled water once the raw materials have been added to the mixing vessel and the rinse water added to the mixing tank.

The above method of preparation may be altered according to the ingredients included in the compositions of the present disclosure. In particular, the order of addition of the optional ingredients may be varied as desired and appropriate. The modifications which may be used include;

Adding the distilled water as a single addition in step 1 so step 9 is omitted.

The at least one monocarboxylic acid, and/or the at least one tricarboxylic acid may be added after the cooling step (step 12).

If the compositions of the present disclosure comprise a foam booster, such as an alkanolamide e.g. cocamide MEA and/or a non-ionic surfactant, such as a glucoside, e.g. decyl glucoside, these are typically added prior to the cooling phase such as after the addition of the amphoteric surfactant.

If the compositions of the present disclosure are formulated as solid compositions, any suitable method of preparation may be used.

The following examples below illustrate exemplary formulations as well as particular embodiments of the present disclosure. It is to be understood that these examples are provided by way of illustration only and that further useful formulations falling within the scope of the present disclosure and the claims may be readily produced by one skilled in the art without deviating from the scope and spirit of the present disclosure.

### EXAMPLES

Compositions according to the present disclosure are provided in the following Examples. The compositions are all intended for application to human skin/hair. They find particular application as cleansers or treatment compositions for human skin and hair. These compositions were prepared following the method of preparation stated hereinabove.

In the following compositions, the ingredients were used “as supplied” from their respective suppliers and some may contain less than 100% wt. of the active material. The ‘active’ amount of each ingredient is indicated in the following tables.

The percentages given in the tables are as % wt. based on the total weight of the compositions. In each of the compositions deionized water was included in “quantum sufficient” (q.s.) to provide 100 parts by weight of the specific composition as stated in the tables.

For each composition in Table 1, the amount of sodium hydroxide solution given is the amount added to bring the composition to its final desired pH.

The compositions above in the tables below 1 were evaluated for their antimicrobial efficacy against Staphylococcus aureus (ATCC 6538) and Escherichia coli (ATCC 10536).

The test method used to evaluate the antimicrobial effectiveness of the Examples was an antimicrobial suspension test based on British standard reference No: EN 1276:2009 method entitled “Chemical Disinfectants and Antiseptics—Quantitative Suspension Test for the Evaluation of bactericidal activity of chemical disinfectants and antiseptics used in food, industrial, domestic and institutional areas—Test method and requirements (Phase 2, step 1)” with the following modifications made to the method; eliminating the interfering substance indicated by the protocol as “dirty or clean conditions” and increasing the test temperature from 20±1° C. to 37±1° C. (as 37° C. is closer to the average human body temperature than 20° C.).

The test protocol tests the efficacy of the compositions of the present disclosure and the comparative example against gram-negative and gram-positive bacteria. The test organisms used were Staphylococcus aureus ATCC 6538 and Escherichia coli ATCC 10536.

The bacterial strains were cultured on tryptic soy agar (TSA) slant from frozen stock and incubated for 24 hours. Following incubation, 2^{nd} and 3^{rd} generation transfers were prepared and used to prepare test suspensions as described in the EN 1276:2009 test method. The cell suspensions were adjusted to produce approximately 1.5−5.0×10⁸ CFU mL⁻¹. The growth medium and temperature used were Tryptic soy Agar (TSA) and 35±1° C. Test solutions and test cultures are equilibrated to a test temperature of 37±1° C. in a water bath.

Experimentally, a 1.0 ml of the test culture was exposed to 9.0 mL of the test product for a 1-minute contact time and neutralized in a verified neutralizer. After a 5-minute neutralization time, the neutralized sample was serially diluted, plated on TSA and incubated at 35=1° C. for 48 hours. The average Log₁₀ CFU/mL for the test suspension was calculated and used to compute the log reduction post-treatment. A three log₁₀ reduction of all tested bacterial strains in a 1-minute contact time was chosen to indicate that the tested formulation has the desired level of antimicrobial properties against tested organism(s).

The formula applied to calculate the log reductions was: Log Nc−Log Nd where: Nc=Number of cfu/ml of the test suspension count and Nd=Number of cfu/ml of the sample count after treatment.

The amounts in the compositions below are given as the % of the active ingredient in the composition unless otherwise stated. The amount of raw material added will therefore be higher than the stated amount for all ingredients which are not 100% active. The amount of raw material added is easily calculated from the information given in the ingredients table below.

TABLE 1
liquid handwash compositions (examples 1 to 6)

| | Ex. 1 | Ex. 2 | Ex. 3 | Ex. 4 | Ex. 5 | Ex. 6 |
|---|---|---|---|---|---|---|
| | % wt | % wt | % wt | % wt | % wt | % wt |
| | active | active | active | active | active | active |
| D.I. water | q.s. | q.s. | q.s. | q.s. | q.s. | q.s. |
| Tetrasodium | 0.087 | 0.087 | 0.087 | 0.087 | 0.087 | 0.087 |
| EDTA | | | | | | |
| Sodium lauryl | 8.4 | 7.0 | 8.4 | 7.0 | 7.0 | 7.0 |
| ether sulfate | | | | | | |
| 2EO (SLES) | | | | | | |
| Cocamide MEA | 0.86 | 0.86 | 0.86 | 0.86 | — | — |
| Cocoamidopropyl | 1.29 | 1.29 | 1.29 | 1.29 | 1.5 | 1.5 |
| betaine | | | | | | |
| Alkyl olefin | — | 0.69 | — | — | — | — |
| sulphonate | | | | | | |
| Lactic acid | 2.15 | 2.15 | 2.0 | 2.5 | 1.5 | 1.5 |
| Citric acid | 0.5 | 0.5 | 0.5 | 0.5 | 1.0 | 1.0 |
| anhydrous | | | | | | |
| Glycerine | 2.0 | 2.0 | 2.0 | 2.0 | 2.0 | 2.0 |
| Sodium chloride | 1.25 | 1.25 | — | — | 0.59 | 0.14 |
| Sodium | 0.47 | 0.47 | — | — | 0.70 | 0.82 |
| hydroxide | | | | | | |
| Sodium benzoate | 0.1 | 0.1 | 0.1 | 0.1 | 0.2 | 0.2 |
| Fragrance | 0.5 | 0.5 | — | — | — | — |
| Ethylene glycol | 2.0 | 2.0 | — | — | — | — |
| monostearate | | | | | | |
| (EGMS) | | | | | | |
| Caprylyl glycol | — | — | — | — | 0.1 | 0.1 |
| Zinc sulphate | — | — | — | — | — | 0.1 |
| heptahydrate | | | | | | |
| Test results | | | | | | |
| pH (final) | | | 3.49 | 3.49 | 4.21 | 4.35 |
| Viscosity cPs | | | | | | 4209 |
| Log₁₀ reduction | | | >4.87 | >5.46 | 3.35 | 5.44 |
| for E. coli (clean) | | | | | | |
| Log₁₀ reduction | | | >5.06 | | 3.39 | 3.52 |
| for S. aureus | | | | | | |
| (clean) | | | | | | |
| Log₁₀ reduction | | | | >5.45 | | |
| for E. coli (dirty) | | | | | | |
| Log₁₀ reduction | | | | >4.02 | | |
| for S. aureus | | | | | | |
| (dirty) | | | | | | |

The content of the sulphonate compound based on the total wt % of the non-sulphonate anionic surfactant and the amphoteric surfactant is 8.3% wt in Example 2.

TABLE 2
liquid handwash compositions (examples 7, 8),
a body wash composition (example 9),
and comparative examples C10 and C11.

| | Ex. 7 | Ex. 8 | Ex. 9 | Ex. C10 | Ex. C11 |
|---|---|---|---|---|---|
| | % wt | % wt | % wt | % wt | % wt |
| | active | active | active | active | active |
| D.I. water | q.s. | q.s. | q.s. | q.s. | q.s. |
| Tetrasodium EDTA | 0.087 | 0.087 | 0.087 | 0.087 | 0.087 |
| Sodium lauryl ether | 7.0 | 7.0 | 10.0 | 7.0 | 7.0 |
| sulfate 2EO (SLES) | | | | | |
| Cocoamidopropyl | 1.5 | 1.5 | 1.5 | 1.5 | 1.29 |
| betaine | | | | | |
| Alkyl polyglucoside | — | — | 0.5 | — | — |
| Cocamide MEA | — | — | 0.5 | — | 0.86 |
| Lactic acid | 1.5 | 1.5 | 1.5 | 1.5 | 2.5 |
| Citric acid anhydrous | 1.0 | 1.0 | 1.0 | 1.0 | — |
| Glycerine | 2.0 | 2.0 | — | 2.0 | — |
| Sodium chloride | 0.2 | 0.2 | 0.3 | 0.23 | — |
| Sodium hydroxide | 0.54 | 0.54 | 0.5 | 0.8 | — |
| Zinc sulphate | — | — | — | — | 0.1 |
| heptahydrate | | | | | |
| Sodium benzoate | 1.0 | 1.0 | 1.0 | 0.2 | 0.1 |
| Fragrance | 0.5 | 0.5 | 0.43 | 0.3 | — |
| Ethylene glycol | 0.5 | 0.5 | 1.0 | 0.5 | — |
| monostearate (EGMS) | | | | | |
| Caprylyl glycol | 0.1 | — | — | 0.1 | 0.4 |
| Para-chloro-meta- | — | — | — | 0.175 | — |
| xylenol (PCMX) | | | | | |
| Dye | 0.02 | 0.02 | — | 0.1 | — |

Test results

| | | | |
|---|---|---|---|
| pH (final) | 4.32 | 4.31 | 3.49 |
| Viscosity cPs | 6299 | 7678 | |
| Log₁₀ reduction for | >5.32 | | >5.46 |
| E. coli.(clean) | | | |
| Log₁₀ reduction for | 3.58 | | |
| S. Aureus. (clean) | | | |
| Log₁₀ reduction for | | | |
| E. Coli.(dirty) | | | |
| Log₁₀ reduction for | | | 3.26 |
| S. Aureus.(dirty) | | | |

TABLE 3
Details of ingredients used in the foregoing Examples.

| Constituent | Function | Details |
|---|---|---|
| D.I. water | Solvent | Distilled water |
| Tetrasodium EDTA | Chelating agent | 87% active solution of tetrasodium |
| | | EDTA |
| Sodium lauryl ether sulfate | Surfactant - anionic | 70% wt. active solution. |
| (SLES) | non-sulfonate | |
| Cocoamidopropyl betaine | Surfactant - | 29% wt. active solution. |
| | amphoteric | |
| Alkyl polyglucoside | Surfactant - nonionic | Decyl glucoside, available as Planteren ™ |
| | | 2000 ex Cognis, 48-55% active. Used as |
| | | 51% active in calculations. |
| Cocamide MEA | Foam booster | 86% active raw material of cocamide |
| | | MEA. |
| Alkyl olefin sulphonate | Surfactant | 92% active raw material of Alkyl olefin |
| | | sulphonate. |
| Lactic acid | Antimicrobial agent | A 50% wt solution of lactic acid |
| Citric acid anhydrous | Antimicrobial agent | Citric acid anhydrous, purity at least |
| | | 99.5% |
| Glycerine | Humectant | Glycerine purity of 99.7% |
| Sodium chloride | Thickener | Sodium chloride purity of 98% minimum |
| Sodium hydroxide | pH adjuster | A 30% solution of sodium hydroxide (98- |
| | | 100% wt. active) |
| Sodium benzoate | Preservative against | Used as the powdered form, available |
| | yeasts and fungi | commercially from a variety of suppliers. |
| Fragrance | fragrance | Proprietary composition of its supplier |
| Ethylene glycol | Pearlizer | Available commercially as Galaxy ™ 610 |
| monostearate (EGMS). | | from Galaxy Surfactants Ltd. |
| Microcare ® CLG | Emollient and aids | 100% wt caprylyl glycol (ex Thor) |
| | preservative effect | |
| Zinc sulphate heptahydrate | Antimicrobial active | Used as the powder. |

The results in Tables 1 and 2 demonstrate that the compositions of the present disclosure show excellent antimicrobial activity on both S. aureus and E. coli. Comparative example C₁₀ contains 0.175% wt PCMX, above the upper limit of 0.15% wt stipulated in claim 1. The excellent antimicrobial activity of the compositions of the present disclosure shows that PCMX at this high level is not needed. Comparative example C₁₁ which contains lactic acid but

### Results no citric acid shows lower activity against S. aureus (dirty) than Example 4 of the present disclosure which contains both lactic acid and citric acid.

The inventors have also noticed that compositions comprising a greater amount of lactic acid than citric acid in combination with the claimed surfactant system are especially advantageous for antimicrobial effects and skin tolerance.

While the present disclosure is susceptible of various modifications and alternative forms, it is to be understood that specific embodiments thereof have been shown by way of example which are not intended to limit the present disclosure to the particular forms disclosed; on the contrary the intention is to cover all modifications, equivalents, and alternatives falling within the scope and spirit of the present disclosure as expressed in the appended claims.

1. A method for providing an antimicrobial benefit to skin and/or hair, comprising the steps of:
contacting an antimicrobial personal care composition with an area of skin and/or hair in a suitable amount and for a sufficient period of time to provide an antimicrobial effect thereon, wherein the antimicrobial personal care composition comprises:
a) 0.1% wt to 20% wt of at least one non-sulfonate anionic surfactant;
b) 0.01% wt to 10% wt of at least one amphoteric surfactant; and
c) at least two carboxylic acids or salts thereof, comprising lactic acid or a salt thereof in an amount of from 0.5% wt to 5% wt and citric acid or a salt thereof in an amount of from 0.25% wt to 4.5% wt, wherein the lactic acid is present in a higher wt % than the citric acid;
wherein the composition contains no more than 30% of the total wt % of a) and b) of sulfonated compounds; and
wherein the composition does not contain para-chloro-meta-xylenol in an amount above 0.15% wt and does not contain salicylic acid in an amount above 0.25% wt; and
wherein the antimicrobial effect comprises effective antimicrobial activity against at least one gram-positive bacteria and at least one gram-negative bacteria.

2. The method according to claim 1, wherein the antimicrobial personal care composition is an aqueous composition and comprises water in an amount of from 65% wt to 99% wt, based on the total weight of the composition.

3. The method according to claim 1, wherein the at least one non-sulfonate anionic surfactant of the antimicrobial personal care composition is selected from the group consisting of alkyl sulfates or salts thereof and alkyl ether sulfates or salts thereof.

4. The method according to claim 3, wherein the alkyl sulfates or salts thereof are selected from the group consisting of C₈-C₁₈ alkyl sulfates and their salts, and wherein the alkyl ether sulfates or salts thereof are selected from the group consisting of C₈-C₁₈ alkyl ether sulfates and their salts.

5. The method according to claim 1, wherein the antimicrobial personal care composition comprises of from 0.5% wt to 15% wt of the at least one non-sulfonate anionic surfactant, based on the total weight of the composition.

6. The method according to claim 1, wherein the amphoteric surfactant of the antimicrobial personal care composition is selected from the group consisting of betaine surfactants represented by the general formula: 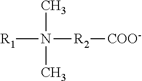
wherein: R₁ is an alkyl group containing from 8 to 18 carbon atoms, or an amido radical which may be represented by the following general formula: 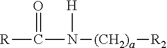
wherein: R is an alkyl group having from 8 to 18 carbon atoms, a is an integer having a value of from 1 to 4 inclusive, and R₂ is a C₁-C₄ alkylene group.

7. The method according to claim 1, wherein the antimicrobial personal care composition is substantially free of at least one of: salicylic acid, sulfonated compounds, para-chloro-meta-xylenol, or combinations thereof.

8. The method according to claim 1, wherein a further carboxylic acid is present in the antimicrobial personal care composition, and wherein the further carboxylic acid is selected from the group consisting of propane-1,2,3-tricarboxylic acid, aconitic acid, agaric acid, trimesic acid, and salts of propane-1,2,3-tricarboxylic acid, aconitic acid, agaric acid, and trimesic acid.

9. The method according to claim 1, wherein a further carboxylic acid is present in the antimicrobial personal care composition, and wherein the further carboxylic acid is selected from the group consisting of acetic acid, glycolic acid, butanoic acid, benzoic acid, nicotinic acid, and salts of acetic acid, glycolic acid, butanoic acid, benzoic acid, and nicotinic acid.

10. The method according to claim 1, wherein the citric acid and lactic acid or salts thereof are present in the antimicrobial personal care composition in a weight ratio in the range of from 1:1.1 to 1:4.

11. The method according to claim 10, wherein the citric acid and lactic acid or salts thereof are present in a weight ratio in the range of from 1:1.25 to 1:3.

12. The method according to claim 11, wherein the citric acid and lactic acid or salts thereof are present in a weight ratio in the range of from 1:1.3 to 1:2.5.

13. The method according to claim 1, wherein the antimicrobial personal care composition has a pH in the range of from about 3.3 to about 5.

14. The method according to claim 1, wherein the antimicrobial personal care composition further comprises a nonionic surfactant in an amount of from 0.01% wt to 15% wt.

15. The method according to claim 1, wherein the antimicrobial personal care composition is selected from the group consisting of a showering or bathing product, a hand washing product, a body care product, a hand care product, a foot care product, a facial care product, a scalp care product, a haircare product, and combinations thereof.

16. The method of claim 1, wherein the antimicrobial effect comprises effective antimicrobial activity against Staphylococcus aureus and effective antimicrobial activity against Escherichia coli.

17. The method of claim 1, wherein the effective antimicrobial activity is defined as at least a 3 log₁₀ reduction in the at least one gram-positive bacteria and at least a 3 log₁₀ reduction in the at least one gram-negative bacteria.

18. The method of claim 1, wherein the area of skin and/or hair is contacted with the antimicrobial personal care composition for a period of at least 10 seconds.

19. The method of claim 1, further comprising the step of rinsing the area of skin and/or hair contacted with the antimicrobial personal care composition.

20. A method for providing an antimicrobial benefit to skin and/or hair, comprising the steps of:
contacting an antimicrobial personal care composition with an area of skin and/or hair in a suitable amount and for a sufficient period of time to provide an antimicrobial effect thereon, wherein the antimicrobial personal care composition comprises:
a) 0.5% wt to 15% wt of at least one non-sulfonate anionic surfactant selected from the group consisting of alkyl sulfates or salts thereof and alkyl ether sulfates or salts thereof;
b) 0.01% wt to 10% wt of at least one amphoteric surfactant selected from the group consisting of betaine surfactants represented by the general formula: 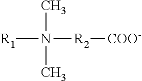
wherein: R₁ is an alkyl group containing from 8 to 18 carbon atoms, or
an amido radical which may be represented by the following general formula: 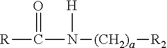
wherein: R is an alkyl group having from 8 to 18 carbon atoms,
a is an integer having a value of from 1 to 4 inclusive, and R₂ is a C₁-C₄ alkylene group; and
c) lactic acid or a salt thereof in an amount of from 0.5% wt to 5% wt and citric acid or a salt thereof in an amount of from 0.25% wt to 4.5% wt, wherein the citric acid and lactic acid or salts thereof are present in a weight ratio in a range of from 1:1.1 to 1:4; and
d) at least one additional carboxylic acid selected from the group consisting of propane-1,2,3-tricarboxylic acid, aconitic acid, agaric acid, trimesic acid, and salts of propane-1,2,3-tricarboxylic acid, aconitic acid, agaric acid, acetic acid, glycolic acid, butanoic acid, benzoic acid, nicotinic acid, and salts of acetic acid, glycolic acid, butanoic acid, benzoic acid, and nicotinic acid;
e) a nonionic surfactant in an amount of from 0.01% wt to 15% wt; and
f) water in an amount of from 65% wt to 99% wt;
wherein the antimicrobial personal care composition is substantially free of at least one of salicylic acid, sulfonated compounds, para-chloro-meta-xylenol, or combinations thereof;
wherein the antimicrobial personal care composition has a pH in the range of from about 3.3 to about 5; and
wherein the antimicrobial effect comprises effective antimicrobial activity against at least one gram-positive bacteria and at least one gram-negative bacteria.
